(19) United States
(12) Patent Application Publication
(10) Pub. No.: US 2011/0223642 A1
(43) Pub. Date: Sep. 15, 2011

(54) METHOD FOR INHIBITING BACTERIA GROWTH DURING ETHANOL FERMENTATION

(76) Inventor: John Paul Maye, Great Falls, VA (US)

(21) Appl. No.: 13/046,362

(22) Filed: Mar. 11, 2011

Related U.S. Application Data
(60) Provisional application No. 61/312,987, filed on Mar. 11, 2010.

(51) Int. Cl. C12P 7/02; A61K 31/122; C12C 11/00

(52) U.S. Cl. 435/155; 514/689; 426/11

(57) ABSTRACT

The present invention is an antibacterial hop product used to inhibit the growth of gram positive bacteria in yeast propagators and ethanol producing fermenters. The hop product is an isomerized or stabilized hop made of alkaline earth metals or alkali earth metals. The antibacterial hop product also enhances the alcohol tolerance of yeast cells.

### CROSS-REFERENCE TO RELATED APPLICATIONS

This Application claims priority from U.S. Provisional Application Ser. No. 61/312,987, filed Mar. 11, 2010, the contents of which are incorporated herein in their entirety.

### 1. TECHNICAL FIELD

The present invention relates to an improved method for inhibiting gram positive bacteria growth and improving the alcohol yields during yeast propagation and ethanol fermentation by treatment with an antibacterial hop product, and to an improved antibacterial hop product, and to a method for making an improved antibacterial hop product. The antibacterial hop product is made by isomerizing alpha acids inside hops into a more water soluble antibacterial isoalpha acid. The antibacterial hop product can be added to a yeast propagator as it relates to yeast or gram negative bacteria growth and/or a fermenter as it relates to ethanol production via yeast or gram negative bacteria. It can also be added to any other stage of the process where gram positive bacterial infection needs to be suppressed.

### 2. BACKGROUND OF THE INVENTION

Fermentable ingredients such as cereals, fruits, starches and canes/beets often arrive from the field contaminated with bacteria. The bacteria concentration can be reduced with washing but not eliminated. High cooking temperatures are often employed to further reduce the bacteria load; however, in some cases this is not practical or it is costly. When these fermentable ingredients are added to a yeast propagator or fermenter the residual bacteria can grow and compete with the yeast for nutrients and fermentable sugars. The result can be a reduction in overall alcohol yield, production of off-flavors or incomplete fermentation. Antimicrobials such as hop acids and other antibiotics often are employed at distilleries to inhibit the growth of gram positive bacteria during yeast propagation and/or fermentation. The gram positive bacteria that commonly infect fermentation streams are lactic acid producing bacteria of the genus Lactobacillus and Pediococcus. Aqueous alkaline solutions of hop acids currently are the only food grade antimicrobial used in distillery mashes; however, they can cost more than antibiotics making them expensive to use. In accordance with prior art practices, these aqueous alkaline solutions are made by extracting hops with CO₂ followed by alpha acid isomerization, isoalpha acid isolation and followed by product formulation. The current invention eliminates the need for CO₂ extraction, isoalpha acid isolation and product formulation. The antibacterial hop product is made by performing solid state chemistry by mixing hop powder with a metal base or oxide followed by pelletization and heating. In addition, most fuel ethanol plants only treat their propagators with an antibacterial. Once that propagator is added to a fermenter, the antibacterial gets diluted to a point where its antibacterial effect is lost. That is not the case with iso-pellets.

### 3. SUMMARY OF THE INVENTION

The present invention solves the need for inhibiting gram positive bacteria growth during yeast propagation and or fermentation cost effectively. Dry hop cones can be ground into a powder and blended with various alkali earth metals or alkaline earth metals or their oxides, bases, carbonates or bicarbonates to form a stabilized hop pellet. The stabilized hop pellets are salts of hop acids, generally salts of alpha acids. By warming these salts, to a temperature of 40° C. to 100° C., preferably about 70° C. to 90° C., more preferably about 80° C., e.g., in a hot room, the alpha acids undergo a thermal isomerization to form isoalpha acids thus forming an isomerized hop pellet also known as iso-pellets. We unexpectedly discovered that by adding antibacterial iso-pellets made in accordance with the present invention we obtained better antibacterial inhibition than by adding an equivalent amount of isoalpha acids alone. While not wishing to be bound by theory, we believe this better antibacterial inhibition is due to a secondary effect caused by other antibacterial compounds in hops such as beta acids and Xanthohumol. In addition it was discovered quite unexpectedly that the yeast became more alcohol tolerant when iso-pellets were used vs. iso-alpha acids alone.

### 4. BRIEF DESCRIPTION OF THE DRAWINGS

The present invention is described herein with reference to the accompanying drawings, in which similar reference characters denote similar elements throughout the several views. It is to be understood that in some instances, various aspects of the invention may be shown exaggerated or enlarged to facilitate an understanding of the invention.

FIG. 1 is a schematic diagram illustrating production of iso-pellets according to the present invention.

FIG. 21A shows a schematic diagram of a typical batch fermentation plant.

FIG. 2B shows a schematic diagram of a typical small fermentation plant, one that does not propagate yeast.

FIG. 2C shows a schematic diagram of a continuous fermentation plant.

FIG. 3 plots extraction of iso-alpha over time at various temperatures.

### 5. DETAILED DESCRIPTION OF THE INVENTION

The present invention is especially suitable for use in forming stabilized hop products useful in fermentation processes, and will be described with such utility.

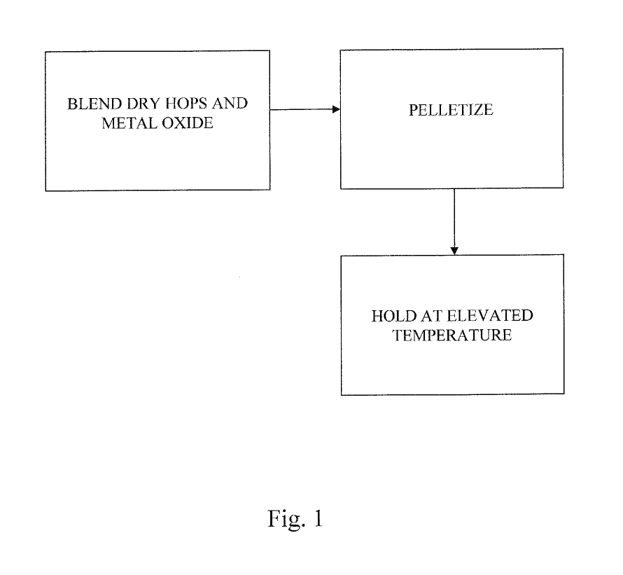

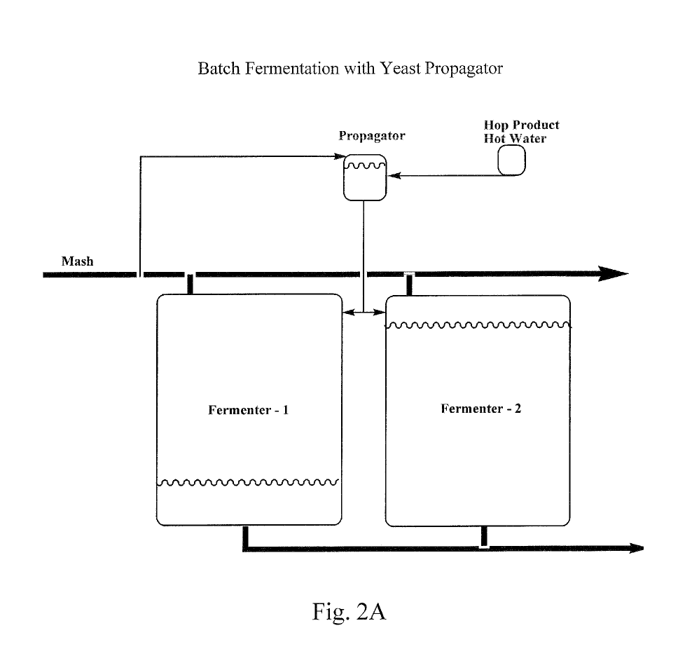

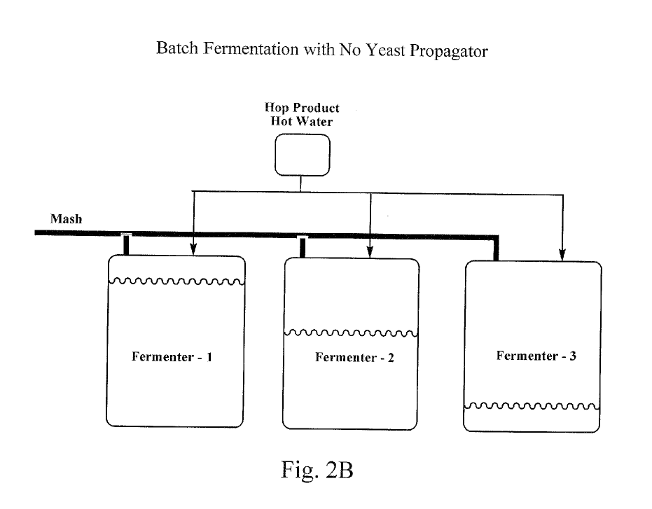

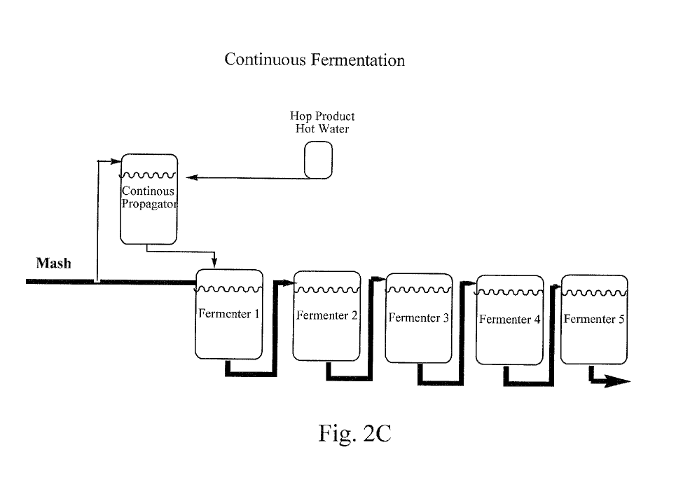

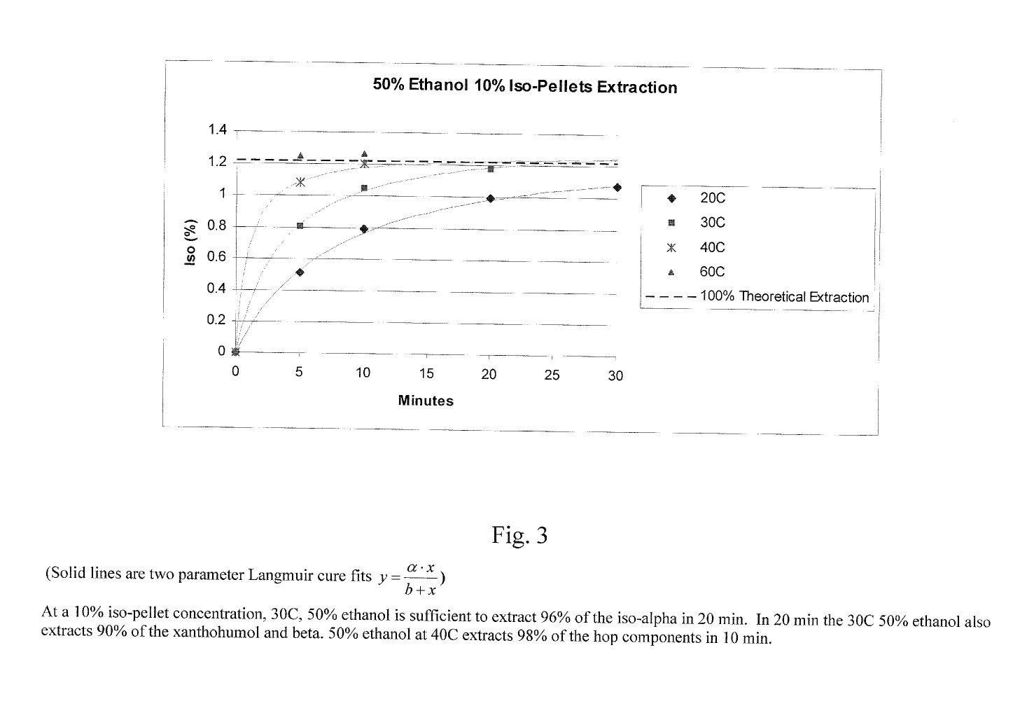

Referring to FIG. 1, dry hop cones are ground into a powder and blended with approximately 0.5 to 10% by weight of a metallic oxide such as calcium oxide or magnesium oxide or a mixture of calcium oxide and magnesium oxide. In a preferred embodiment the metallic oxide comprises magnesium oxide, preferably about 4% by weight magnesium oxide. However, as noted supra other alkali or alkaline earth metals or their oxides, bases, carbonates or bicarbonates may be used. The blend is pelletized, and held under slightly elevated temperature, e.g., heated in a hot room at 55° C. for 8 to 12 days to form an isomerized hop pellet. It has been found that with the use of alkali or alkaline earth metal materials as above described in the isomerizing process, the moisture or water naturally present in hops, typically from about 6 to about 15% by weight, is sufficient to cause a transformation in most of the alpha acids present to their isomerized form, i.e. iso-alpha acids.

The resulting isomerized hop pellets are not very soluble in cold or warm water; however they are very soluble in hot water of temperatures over 50° C., preferably over 70° C. Once the isomerized hop pellets are dissolved in hot water they can be added to a yeast propagator and or fermenter at a dose rate of approximately 20 ppm to 300 ppm, preferably about 125 ppm based on 12% isoalpha acids. If added just to a propagator the isopellets should be diluted down to 2 to 20 ppm in a fermenter. The hop acids employed are not limited to isomerized hop acids but can also be stabilized unisomerized hop pellets. The stabilized hop pellet, can be isomerized into isoalpha acids once placed into 70° C. or hotter hot water for thirty or more minutes. Once this isomerization is completed the mixture can be added to the yeast propagator or fermenter. The unexpected results include reduced levels of isoalpha acid required to inhibit gram positive bacteria growth, better antibacterial protection toward the end of fermentation, and increased yeast alcohol tolerance. Typically about 15 ppm of isoalpha acid is required to inhibit most gram positive bacteria growth in a typical corn mash media; however, we discovered that isomerized hop pellets made in accordance with the present invention can inhibit bacteria growth at an isoalpha acid dosage of about 12 ppm. While not wishing to be bound by theory, it is believed iso-pellets made in accordance with the present invention also contain beta acids, which are very effective at inhibiting gram positive bacteria growth. Unfortunately beta acids ordinarily are not soluble in corn mash or cereal fermentations, and thus their antibacterial effect is worthless. Surprisingly, we unexpectedly discovered that as the fermentation proceeds and the alcohol yields increase, the alcohol solubilizes the beta acids allowing it to inhibit bacteria growth toward the end of fermentation. This unexpected benefit allows one to inhibit bacteria growth at the start of fermentation with iso-alpha acids and at the end with beta acids making iso-pellets more cost effective to use. This observation also leads to the discovery that if we dissolve the iso-pellets in a water/alcohol solution, it dissolves essentially all the actives in the iso-pellets, whereas with water only, it seems to dissolve essentially only just the iso-alpha acids. Preferably the alcohol comprises a lower alcohol, e.g. containing 1 to 6 carbon atoms, more preferably, methanol, ethanol, propanol or the like, or a mixture of lower alcohols. Of course, in the case of a fermentation process for producing a food product or beverage, the alcohol should be food compatible. Generally, the alcohol is present in an amount of 20% to 80% w/w alcohol/water, more preferably 40% to 60% w/w alcohol/water, most preferably 50%% w/w alcohol/water. Another unexpected benefit was increased alcohol tolerance of the yeast. Hops contain polyphenols and prenylasted flavanoids which are known to have antioxidant properties. Many fuel ethanol plants have difficulty achieving alcohol yields higher than about 17.5% w/w due to alcohol stress. Advantageously, the antioxidant properties of hop polyphcnols extends yeast life allowing the yeast to produce alcohol yields consistently higher than one would normally experience.

For clarity of disclosure, and not by way of limitation, the detailed description of the invention is divided into the subsections set forth below.

5.1. How The Antibacterial Isomerized Hop Pellet is Used

Referring to FIG. 2A, antibacterial hop pellets made in accordance with the present method, as above described, can be used in a batch fermentation plant. This could be a brewery, winery, spirit plant (distillery), fuel ethanol plant or even a pharmaceutical plant. In FIG. 2A there is only one propagator and two fermenters shown. However, some plants have more than one propagator and most have more than two fermenters. At these plants, mash containing fermentable sugars are used to fill a propagator as well as the fermenters. In a separate tank, hot water, greater than 70° C. is added to the iso-pellets to make a 1 to 20% w/w solution of iso-pellets. Active dry yeast such as Saccharomyces cerevisiae is added to the propagator along with the antibacterial iso-pellet solution to achieve an isoalpha acid concentration greater than 10 ppm up to 60 ppm preferably about 12 ppm. Air preferably is bubbled within the propagation tank to help the yeast grow. Over a period of about 8 to 15 hours the yeast in the propagator will utilize the sugars from the mash for growth. Once the yeast reaches the expected cell concentration, it is pumped from the propagator to a fermenter just being filled, e.g. Fermenter 1, thus diluting the iso-pellet concentration to 2 to 20 ppm.

Referring now to FIG. 2B, some small fermentation plants do not propagate their yeast. This could be a small brewery, winery, distillery or fuel ethanol plant. In this case active dry yeast, Saccharomyces cerevisiae, may be added directly to a fermenter as it is being filled. All the other ingredients for the fermentation are also added such as enzymes, nutrients and the antibacterial hop product to achieve an isoalpha acid concentration greater than 10 ppm up to 60 ppm preferably 12 ppm.

Referring now to FIG. 2C, unlike batch fermentation plants, continuous fermentation plants never empty any of their tanks. In fact, a small amount of fermentable mash is constantly being fed into an aerated tank called a prefermenter or continuous propagator. The time the yeast spends growing in the prefermenter is dependent on the flow rate of the mash going in and out of that tank. The flow rate is adjusted so that the yeast replaces itself at a rate equivalent to the amount of yeast being pumped out of the propagator. The yeasted mash in the prefermenter will be continuously pumped from one fermentation tank to the next, e.g. first into Fermenter 1 and the contents of Fermenter 1 will be continuously pumped into Fermenter 2 and the contents of Fermenter 2 will be continuously pumped into Fermenter 3, and so on. This series of fermentation tanks is often referred to as a fermentation train. In order to achieve very low gram positive bacteria cell counts in a continuous propagator and in the fermentation train the antibacterial hop product should be added continuously via a metering pump into the top of the propagation tank to achieve an isoalpha acid concentration greater than about 10 ppm but less than about 100 ppm or preferable about 80 ppm.

The following examples are offered by way of illustration and not by way of limitation.

### EXAMPLES

### Example 1

### Improving the Alcohol Tolerance of Yeast Using Isomerized Hop Pellets

A trial was conducted at a batch fermentation, dry mill, fuel ethanol plant comparing the alcohol tolerance alcohol yield of fermenters treated with a purified solution of isoalpha acids vs. Iso-pellets. 4-kg of 30% Iso-alpha acid (containing 70% water) were added to each fermenter and compared to fermenters treated with an equivalent amount of isoalpha acids via iso-pellets (10-kg of Iso-Pellets containing 12% isoalpha acids). The trial was conducted over multiple fermentations and in each an every case, the Iso-pellet treated fermenters showed on average a 0.1% increase in alcohol yield vs. the 30% isoalpha acid treated fermenters. Yeast will only produce more alcohol if it is more tolerant to alcohol. We believe this increase in alcohol yield is due to the antioxidant polyphenols compounds found in Iso-pellets as well as the enhanced antibacterial effect that comes from the other compounds found within Iso-Pellets like beta acids and xanthohumol. That increase in alcohol yield is equivalent to 750 additional gallons per fermenter based on a fermenter size of 750,000 gallons. Therefore a typical 50 million gallon fuel ethanol plant could experience 375,000 gallons of additional ethanol per year by using Iso-pellets vs 30% Iso-alpha acids.

### Example 2

### Procedure

5.00 g of P-249-09 iso-pellets, made as in Example 1 where placed in a 100 mL beaker. To the pellets, 45.0 g of 50% ethanol was added at 20 C, 30 C, 40 C and 60 C to make a 10% Iso-Pellet Slurry. The pellets were stirred with a Teflon stir bar and the temperature was maintained within IC. 2.5 g aliquots were filtered through a 0.45 μm syringe filter with a cotton pre-filter at 5, 10, 20, and 30 min; then diluted to 50 mL with acidic methanol for HPLC analysis.

Results are reported in Table 2 and Plotted in FIG. 3. As can be seen from Table 2 and FIG. 2, adding ethanol to water increases extraction to close to theoretical.

TABLE 2

| | Xantholhumol | Iso | Alpha | Beta |
|---|---|---|---|---|
| | % | (%) | (%) | (%) |
| P-294-09 | 0.64 | 11.0 | 0.34 | 3.12 |
| Iso-Pellets | | | | |
| 100% Extraction | 0.071 | 1.22 | 0.038 | 0.35 |

TABLE 3

| Temperature | | Xanthohumol | Iso | Alpha | Beta |
|---|---|---|---|---|---|
| (C.) | Time | (%) | (%) | (%) | (%) |
| 60 | 5 | 0.072 | 1.25 | 0.037 | 0.356 |
| | 10 | 0.072 | 1.26 | 0.031 | 0.364 |
| 40 | 5 | 0.063 | 1.08 | 0.041 | 0.276 |
| | 10 | 0.069 | 1.20 | 0.044 | 0.311 |
| 30 | 5 | 0.045 | 0.81 | 0.018 | 0.216 |
| | 10 | 0.057 | 1.05 | 0.023 | 0.283 |
| | 20 | 0.063 | 1.17 | 0.027 | 0.313 |
| 20 | 5 | 0.031 | 0.51 | 0.019 | 0.130 |
| | 10 | 0.017 | 0.79 | 0.028 | 0.205 |
| | 20 | 0.058 | 0.99 | 0.034 | 0.259 |
| | 30 | 0.061 | 1.07 | 0.036 | 0.297 |

Other modifications and variations of this invention will occur to those skilled in the art in the light of the above description. It is to be understood, therefore, that changes may be made in the particular embodiments described herein which are within the full intended scope of the invention as defined in the appended claims.

What is claimed is:

1. An antibacterial hop product comprising a stabilized hop or isomerized hop product containing alpha acids, isoalpha acids, beta acids or mixtures thereof.

2. The hop product of claim 1 in the form of a powder or a pellet.

3. The hop product of claim 1, wherein the stabilized hop or isomerized hop product is made from an alkali or alkaline earth metal, or an oxide, base, carbonate or bicarbonate thereof, or mixtures thereof.

4. The hop product of claim 3, wherein the alkali metal oxide is selected from the group consisting of magnesium oxide, calcium oxide and a mixture thereof.

5. The hop product of claim 1, wherein the product is predissolved in an aqueous or aqueous-alcohol solution.

6. The hop product of claim 5, wherein the alcohol comprises a lower alcohol having 1 to 6 carbon atoms.

7. The hop product of claim 6, wherein the alcohol comprises ethanol.

8. A method for inhibiting bacteria growth and improving alcohol yield during yeast propagation and alcohol fermentation, which comprises adding the antibacterial hop product of claim 1 to the yeast propagator and/or fermenter.

9. The method of claim 8, wherein the antibacterial hop product is dissolved in a water solution prior to addition to the yeast propagator or fermenter, and added at a dose rate of 50 to 300 ppm based on an isoalpha acid content of 12%.

10. The method of claim 9, wherein the antibacterial hop product added a dose rate of 100 to 200 ppm based on an iso-alpha acid content of 12%.

11. The method of claim 9, wherein the water solution also includes a lower alcohol.

12. The method of claim 11, wherein the lower alcohol comprises 1 to 6 carbon atoms.

13. The method of claim 12, wherein the lower alcohol comprises ethanol.

14. The method of claim 8, wherein the bacteria comprises gram positive bacteria.

15. The method of claim 8, wherein the fermenter comprises a batch fermentation plant.

16. The method of claim 8, wherein the fermenter comprises a continuous fermentation plant.

17. The method of claim 8, wherein the fermenter comprises a fuel ethanol plant.

18. The method of claim 8, wherein the fermenter comprises a food or beverage fermenter
